(19) United States
(12) Patent Application Publication
(10) Pub. No.: US 2015/0265584 A1
(43) Pub. Date: Sep. 24, 2015

(54) LEVUGLANDIN ADDUCTS HISTONE H4 IN A CYCLOOXYGENASE-2-DEPENDENT MANNER, ALTERING ITS INTERACTION WITH DNA

(71) Applicant: VANDERBILT UNIVERSITY, Nashville, TN (US)

(72) Inventors: John A. Oates, Nashville, TN (US); Olivier Boutaud, Nashville, TN (US); Venkataraman Amarnath, Nashville, TN (US); Erica J. Carrier, Nashville, TN (US)

(21) Appl. No.: 14/662,080

(22) Filed: Mar. 18, 2015

Related U.S. Application Data
(60) Provisional application No. 61/954,827, filed on Mar. 18, 2014.

(51) Int. Cl. A61K 31/44; A61K 31/198; A61K 31/135

(52) U.S. Cl. CPC A61K 31/44; A61K 31/135; A61K 31/198

(57) ABSTRACT

Method of inhibiting formation of levuglandin adducts of histone and DNA in a subject in need thereof by administering a levuglandin adduct formation inhibiting amount of a compound of the following formula:

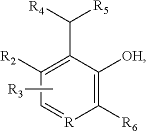

wherein the variables are defined herein.

### PRIOR APPLICATION INFORMATION

This application claims benefit to U.S. Patent Application 61/954,827, filed Mar. 18, 2014, the contents of which are incorporated herein by reference.

### GOVERNMENT SUPPORT

This invention was made with government support under P30 ES000267, P50 CA90949 and P50 GM 15431 awarded by the National Institutes of Health. The government has certain rights in the invention

### BACKGROUND OF THE INVENTION

Embodiments of this invention relate to methods of inhibiting the modification of histones and DNA by levuglandins in a subject in need thereof by administering a compound of the present invention or a pharmaceutically acceptable salt thereof.

### SUMMARY OF THE INVENTION

Inflammation and subsequent cyclooxygenase-2 (COX-2) activity has long been linked with the development of cancer, although little is known about any epigenetic effects of COX-2. A product of COX-2 activity, levuglandin (LG) quickly forms covalent bonds with nearby primary amines, such as those in lysine, which leads to LG-protein adducts. Here, we demonstrate that COX-2 activity causes LG-histone adducts in cultured cells and liver tissue, detectable through LC/MS, with the highest incidence in histone H4. Adduction is blocked by a γ-ketoaldehyde scavenger, which has no effect on COX-2 activity as measured by PGE₂ production. Formation of LG-histone adduct is associated with an increased histone solubility in NaCl, indicating destabilization of the nucleosome structure; this is also reversed with scavenger treatment. These data demonstrate that COX-2 activity can cause histone adduction and loosening of the nucleosome complex, which could lead to altered transcription and contribute to carcinogenesis.

Accordingly, embodiments of the present invention include a method of inhibiting formation of levuglandin adducts of histone and DNA in a subject in need thereof by administering a compound of the present invention or a pharmaceutically acceptable salt thereof.

Another embodiment of the present invention is a method of treating pre-malignant lesions by administering a compound of the present invention or a pharmaceutically acceptable salt thereof.

Another embodiment of the present invention is a method of scavenging levuglandins in nucleus of cells by administering a compound of the present invention or a pharmaceutically acceptable salt thereof.

Another embodiment of the present invention is preventing malignant mutations of pre-cancerous conditions by administering a compound of the present invention or a pharmaceutically acceptable salt thereof.

Another embodiment of the present invention is decreasing a subject's risk of developing cancer by administering a compound of the present invention or a pharmaceutically acceptable salt thereof.

Another embodiment of the present invention is the prevention of cellular transformation to malignancy by administering a compound of the present invention or a pharmaceutically acceptable salt thereof.

Another embodiment of the present invention is a method preventing further mutations in colon, esophagus, breast, lung, pancreas and/or prostate cancers in a subject in need thereof by administering a compound of the present invention or a pharmaceutically acceptable salt thereof.

Another embodiment of the present invention is a method treating and/or preventing a disorder resulting from elevated levels of LG-histone adduct formation.

Another embodiment of the present invention is when the disorder resulting from elevated levels of LG-histone adduct formation is neoplasia. In other embodiments, the neoplasia is a brain cancer, a bone cancer, an epithelial cell-derived neoplasia (epithelial carcinoma), a basal cell carcinoma, an adenocarcinoma, a gastrointestinal cancer, a lip cancer, a mouth cancer, an esophageal cancer, a small bowel cancer, a stomach cancer, a colon cancer, a liver cancer, a bladder cancer, a pancreas cancer, an ovary cancer, a cervical cancer, a lung cancer, a breast cancer, a skin cancer, a squamus cell cancer, a basal cell cancer, a prostate cancer, a renal cell carcinoma, a cancerous tumor, a growth, a polyp, an adenomatous polyp, a familial adenomatous polyposis or a fibrosis resulting from radiation therapy.

Another embodiment of the present invention relates to treatment of a disease such as especially pre-malignant lesions of the colon or esophagus (Barrett's esophagus) or a colon cancer or other malignancies, preferably pre-malignant colon lesions or a colon cancer, in a subject in need thereof. The other malignancies to be treated according to the present invention are preferably selected from the group consisting of breast cancer, lung cancer, ovarian cancer, lymphoma, head and neck cancer and cancer of the esophagus, stomach, bladder, prostrate, uterus and cervix.

Another embodiment of the present invention is a commercial package or product comprising a LG-histone adduction inhibitor, in particular those mentioned herein, or a pharmaceutically acceptable salt thereof, together with instructions for the treatment of a disease such as especially pre-malignant colon lesions or a colon cancer or other malignancies, preferably pre-malignant colon lesions or a colon cancer, in subject in need thereof.

According to the present invention, a patient is treated with therapeutically effective amounts of a LG-histone adduction inhibitor of the present invention, each according to a dosage regimen that is appropriate for the individual agent. For example, LG-histone adduction inhibitor may be administered once or more daily, on alternate days or on some other schedule—as is appropriate. One of skill in the art has the ability to determine appropriate pharmaceutically effective amounts of the combination components.

In the context of the present invention the terms “treatment” or “treat” refer to both prophylactic or preventative treatment as well as curative or disease modifying treatment, including treatment of patients at risk of contracting the disease or suspected to have contracted the disease as well as patients who are ill or have been diagnosed as suffering from a disease or medical condition.

### BRIEF DESCRIPTION OF THE DRAWINGS

FIG. 1 shows a scheme and structure of the LG-lysyl adduct and the fragment ions monitored in positive ion mode (+H).

FIG. 2 is a series of graphs showing that LG-lysine adducts of histones are found in cells and tissue, dependent on COX-2 activity. RAW264.7 mouse macrophage (A) and A549 human lung carcinoma (C) cells were stimulated to express COX-2, then given 20 μM arachidonic acid (AA) or vehicle. A subgroup of cells was preincubated 45 min with 50 μM indomethacin. As a measure of COX activity, PGE₂ was determined by GC/MS from cell media prior to lysis (B and D). Nuclei were isolated, and histones were extracted and digested to individual amino acids prior to LC/ES/MS/MS analysis. *, p<0.05; ***, p<0.001 by ANOVA followed by Tukey's post-test (n≧5). (E). Histones were extracted from nuclei of rat liver, and analyzed as above for LG-lactam adduct. COX-2 protein was analyzed by Western blotting and plotted against lactam adduct levels. Each point corresponds to 1 liver, and shown is the line of regression (r²=0.7237). Pearson r=0.8507; two-tailed p=0.0152. (F) LC-MS chromatograph of histones isolated from a rat liver with relatively high COX-2 expression (COX-2 band intensity of 117 arbitrary units).

FIG. 3 shows LG-lysyl adducts are predominantly detected on histone H4. (A). A Ponceau stain of a sample A549 histone extraction is shown, along with band identities. Histones were extracted from nuclei in 0.4N H₂SO₄, resolved on 4-12% SDS-PAGE gradient gel and transferred to nitrocellulose. H3 and H2B tend to run together as one band. (B). RAW264.7 or A549 cells were stimulated to express COX-2 and given 20 μM ¹⁴C-AA for 1 h. Cells were lysed, nuclei were isolated, and histones extracted, concentrated, and resolved on SDS-PAGE prior to transferring to nitrocellulose and exposing to film. Shown is the Coomassie stain of the SDS-PAGE gel (left) and the result of autoradiography (right). The present inventors have observed that Ponceau, Coomassie R-250, Coomassie G-250, and silver stains each preferentially detect different histone or acid-soluble proteins. (C-D). RAW264.7 (C) or A549 (D) cells were stimulated to express COX-2, and treated with 20 μM AA for 1 h prior to histone extraction. 350-400 μg of total histone was loaded onto 4-12% SDS-PAGE gel and transferred to nitrocellulose. Individual bands were excised horizontally and proteins digested directly off the nitrocellulose by serial incubations with Pronase and aminopeptidase. The results were analyzed by LC/ESI/MS/MS, and the chromatographs of the H3/H2B and H4 bands shown against the LG-lysyl internal standard. The H2A chromatograph is shown as a representative negative result; no co-migrating peaks were seen in any other bands.

FIG. 4 shows that the scavenger EtSA blocks LG-lysyl adduct formation in RAW264.7 and A549 histones, without affecting COX-2 activity. (A). Scavengers were screened in RAW264.7 cells for the ability to decrease LG adduct formation on histones. Scavengers used were glucosamine (GA), 3-methoxysalicylamine (3-MoSA), pentylpyridoxamine (PPM), and 5-ethylsalicylamine (EtSA). Cells were stimulated to express COX-2, pretreated 45 min. with 500 μM scavenger or vehicle (H₂O), and given 20 μM AA for 1 h before lysing and extracting histones. Histone proteins were analyzed by LC/ESI/MS/MS for LG-lysyl lactam adduct, n=2. (B) Stimulated A549 cells were pretreated with 30, 300, or 1000 μM EtSA prior to 1 h with 20 μM AA, and histones analyzed for LG-lysyl adduct. *, p<0.05 by one-way ANOVA followed by Dunnett's multiple comparisons post-test (n=3-5). (C). A549 cells were stimulated, pretreated 45 min. with 1000 μM EtSA or H₂O vehicle, and given 20 μM AA for 1 h. Media was analyzed by GC/MS for PGE₂ (n=3). There was no effect on PGE₂ production at lower doses of EtSA (data not shown).

FIG. 5. LG-lysyl adduct formation on histone H4 decreases DNA-histone interaction. A549 cells were stimulated and given DMSO vehicle (C lanes) or 20 μM AA for 1 h (A lanes). A subgroup of cells was treated with 500 μM EtSA 45 min prior to adding AA (E lanes). Nuclei were extracted with 0.6, 0.9, or 1.2 M NaCl buffer, and the supernatant evaluated by Western blotting for histone H4. Shown is a representative Western blot (A) as well as the pooled results of 4 experiments (B). Different exposure times may have been used for the 0.9 M and 1.2 M bands. ***, p<0.001 by one-way ANOVA followed by Tukey's multiple comparisons post-test. NS, not significant.

### DESCRIPTION OF THE INVENTION

The present invention can be understood more readily by reference to the following detailed description of the invention and the Examples included therein.

Before the present compounds, compositions, articles, systems, devices, and/or methods are further disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which need to be independently confirmed.

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “optional” or “optionally” means that the subsequently described event or circumstance can or cannot occur, and that the description includes instances where said event or circumstance occurs and instances where it does not.

As used herein, the term “levuglandin scavenger” is a compound that prevents reactive carbonyls such as levuglandin from reacting with DNA and proteins. Without being bound by theory or mechanism, this may occur by reacting with the carbonyls to form covalent adducts, thus preventing them from forming adducts of DNA and proteins.

Cyclooxygenase-2 (COX-2) expression is associated with the development of many cancers, and the enzyme plays a key role in the progression of chronic gastrointestinal inflammation to cancer. Predictably, treatment with COX inhibitors decreases a person's total risk of cancer. Prevention studies as well as animal models suggest that increased COX-2 activity is both an early event in carcinogenesis, which contributes to the cellular transformation to malignancy, as well as a sustained event in some colorectal and lung cancers that can be associated with metastasis and poorer clinical prognosis. As predicted by these data, inhibiting COX-2 activity with non-steroidal anti-inflammatory drugs (NSAIDS) or COX-2-specific inhibitors over time reduces a person's total risk of colon, breast, lung, and prostate cancers.

Despite the promise of these drugs in cancer prevention, the gastrointestinal toxicity associated with long-term NSAID treatment and increased cardiovascular events associated with COX-2-specific inhibitors limit their clinical use. A better understanding of the specific downstream contributions of COX-2 to carcinogenesis could lead to new treatments that bypass these undesirable effects.

The product of COX-2, prostaglandin H₂ (PGH₂), is converted enzymatically into other prostaglandins, and indeed PGE₂ is a well-described promoter of carcinogenesis. However, depending on the animal model, deletion of microsomal PGE₂ synthase-1 can either prevent or accelerate tumorigenesis, indicating that the contribution of COX-2 to cancer, particularly to cellular transformation, is probably multifaceted.

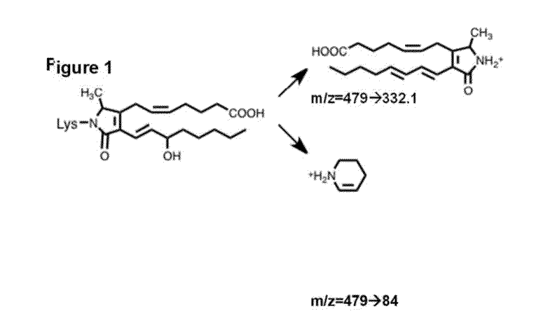

Besides enzymatic conversion, PGH₂ also spontaneously rearranges in aqueous solution to form the highly reactive levuglandins, LGE₂ and LGD₂. The γ-ketoaldehyde levuglandins (LGs) constitute about 20% of total PGH₂ rearrangement products. Newly formed LGs react almost immediately with free amino groups, such as those in lysine, which leads to stable covalent LG-protein adducts measureable by mass spectrometry (FIG. 1) or protein-protein crosslinks. Following COX-2 activity, LG adducts of protein form in cells and in tissues. Proteins rich in lysine are thus especially susceptible to adduction, and due to the perinuclear localization of COX-2 and a PGH₂ half-life measured in minutes, we speculated that it would be possible for PGH₂ to cross the nuclear envelope before rearranging to LGE₂, allowing formation of LG adduct on the lysine-rich histones.

The present inventors have discovered LG-histone adducts in multiple cancer cell lines as well as rat liver, with highest measurable amounts of adduct on the H4 histone. Adduct formation is dependent on COX-2 expression or activity. Lysines, with their short sidechain and positive charge, are critical for histone ionic interaction with DNA, and the inventors find that this interaction is decreased with the introduction of a hydrophobic negative charge from LG. Covalent histone modifications are a major method of controlling gene expression. Changes to lysyl modifications of histones are associated with human cancer. These findings link COX-2 induction with perturbation of normal DNA-histone interactions and provide a novel role for the enzyme in carcinogenesis.

Importantly, the inventors have found a small-molecule salicylamine derivative that scavenges LG to reduce adduct formation on histones without affecting COX-2 activity. In comparison with other scavengers, salicylamine, pyridoxamine and their other analogues, ethylsalicylamine was most potent in protecting histones from modification by levuglandins, indicating that it has the key property of being transported into the nucleus.

### Compounds

It is understood that the following disclosed compounds can be employed in the disclosed methods of using or treating.

Examples of these compounds include, but are not limited to, compounds selected from the formula:

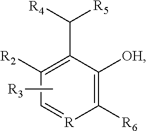

wherein:

R is N or CR₄;

R₂ is H, substituted or unsubstituted alkyl, substituted or unsubstituted alkoxy, alkyl-alkoxy-R₄;

R₃ is H, halogen, substituted or unsubstituted alkyl, substituted or unsubstituted alkoxy, hydroxyl, nitro;

R₄ is a bond, H, substituted or unsubstituted alkyl, substituted or unsubstituted alkoxy, carboxyl, substituted or unsubstituted aryl, or substituted or unsubstituted cycloalkyl:

R₅ is amine;

R₆ is H, substituted or unsubstituted alkyl, substituted or unsubstituted alkoxy; or analogs thereof; and pharmaceutical salts thereof.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, optionally substituted alkyl, cycloalkyl, alkoxy, amino, ether, halide, hydroxy, nitro, silyl, sulfo-oxo, or thiol as described herein.

The term “alkoxy” group includes an alkyl group as defined above joined to an oxygen atom having preferably from 1 to 4 carbon atoms in a straight or branched chain, such as, for example, methoxy, ethoxy, propoxy, isopropoxy (1-methylethoxy), butoxy, tert-butoxy(1,1-dimethylethoxy), and the like.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

The terms “amine” or “amino” as used herein are represented by a formula NA¹A²A³, where A¹, A², and A³ can be, independently, hydrogen or optionally substituted alkyl, cycloalkyl, alkenyl, cycloalkenyl, alkynyl, cycloalkynyl, aryl, or heteroaryl group as described herein.

Examples of compounds or analogs of the present invention include compounds of the following formula:

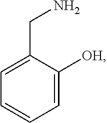

or an analog thereof, and pharmaceutical salts thereof.

Further examples of compounds or analogs of the present invention include compounds of the following formula:

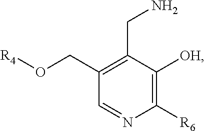

or an analog thereof, and pharmaceutical salts thereof.
Further examples of compounds or analogs of the present invention include compounds of the following formula:

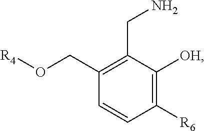

or an analog thereof, and pharmaceutical salts thereof.

Further examples of compounds or analogs of the present invention include compounds of the following formula:

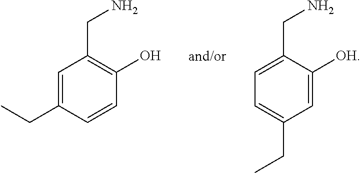

Further compounds or analogs may also be chosen from:

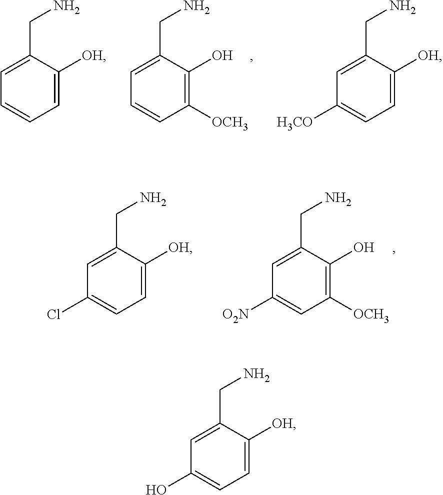

or an analog thereof.

The compounds may also be chosen from:

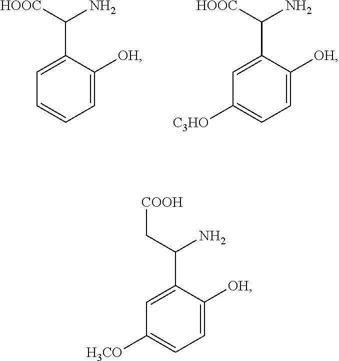

or an analog thereof.

The compounds of the present invention can also be chosen from:

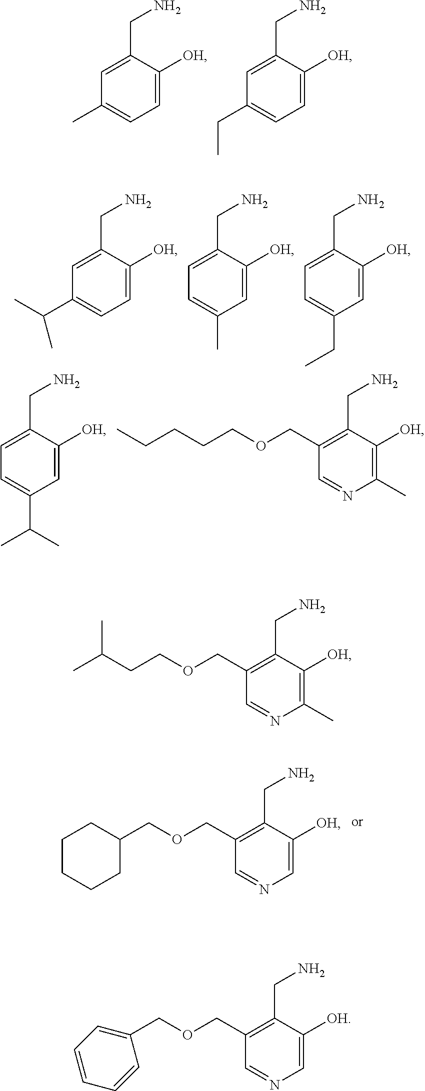

The compounds of the present invention can be administered as the sole active pharmaceutical agent, or can be used in combination with one or more other agents useful for treating or preventing various complications, such as, for example, lesions and cancer. When administered as a combination, the therapeutic agents can be formulated as separate compositions that are given at the same time or different times, or the therapeutic agents can be given as a single composition.

The compounds of the present invention may be made up in a solid form (including granules, powders or suppositories) or in a liquid form (e.g., solutions, suspensions, or emulsions). They may be applied in a variety of solutions and may be subjected to conventional pharmaceutical operations such as sterilization and/or may contain conventional adjuvants, such as preservatives, stabilizers, wetting agents, emulsifiers, buffers etc.

For administration, the compounds of the present invention are ordinarily combined with one or more adjuvants appropriate for the indicated route of administration. For example, they may be admixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, and tableted or encapsulated for conventional administration. Alternatively, they may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

In therapeutic applications, the compounds of the present invention may be administered to a patient in an amount sufficient to reduce or inhibit the desired indication. Amounts effective for this use depend on factors including, but not limited to, the route of administration, the stage and severity of the indication, the general state of health of the mammal, and the judgment of the prescribing physician. The compounds of the present invention are safe and effective over a wide dosage range. However, it will be understood that the amounts of compound actually administered will be determined by a physician, in the light of the above relevant circumstances.

The compounds of the present invention may be administered by any suitable route, including orally, parentally, by inhalation or rectally in dosage unit formulations containing conventional pharmaceutically acceptable carriers, adjuvants, and vehicles, including liposomes. The term parenteral as used herein includes, subcutaneous, intravenous, intraarterial, intramuscular, intrastemal, intratendinous, intraspinal, intracranial, intrathoracic, infusion techniques, intracavity, or intraperitoneally. In a preferred embodiment, ethylsalicylamine is administered orally or parentally.

Pharmaceutically acceptable acid addition salts of the compounds suitable for use in methods of the invention include salts derived from nontoxic inorganic acids such as hydrochloric, nitric, phosphoric, sulfuric, hydrobromic, hydriodic, hydrofluoric, phosphorous, and the like, as well as the salts derived from nontoxic organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate, galacturonate, n-methyl glutamine, etc. (see, e.g., Berge et al., J. Pharmaceutical Science, 66: 1-19 (1977).

The acid addition salts of the basic compounds are prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

### EXAMPLES

The following examples and discussion are to be construed as being exemplary of the present invention, and not intended to be limiting thereof.

### Experimental Procedures

### Materials

All reagents and chemicals were purchased from Sigma-Aldrich (St Louis, Mo.) unless otherwise noted. Methanol and acetonitrile were from Fisher Scientific (Pittsburgh, Pa.) and were HPLC grade or higher. [14C]-arachidonic acid (AA) was obtained from Perkin-Elmer Life Sciences (Boston, Mass.). The following γ-ketoaldehyde scavenger molecules were synthesized by V. Amamath as previously described: pentylpyridoxamine (PPM), 3-methoxysalicylamine (3-MoSA), and 5-ethylsalicylamine (EtSA).

### Treatment of Cells

To stimulate COX-2 expression, A549 or RAW264.7 cells were treated overnight with 5 ng/mL IL-1β (A549) or 10 μg/mL LPS and 10 U/mL IFNγ (RAW264.7) in serum-free medium. When indicated, cells were pretreated with indomethacin, aldehyde scavengers (glucosamine, 3-MoSA, PPM or EtSA), or vehicle (ethanol for indomethacin, H₂O for scavengers) for 45 min, and then given 20 μM arachidonic acid (AA) or DMSO vehicle for 1 h before lysing.

### Histone Extraction

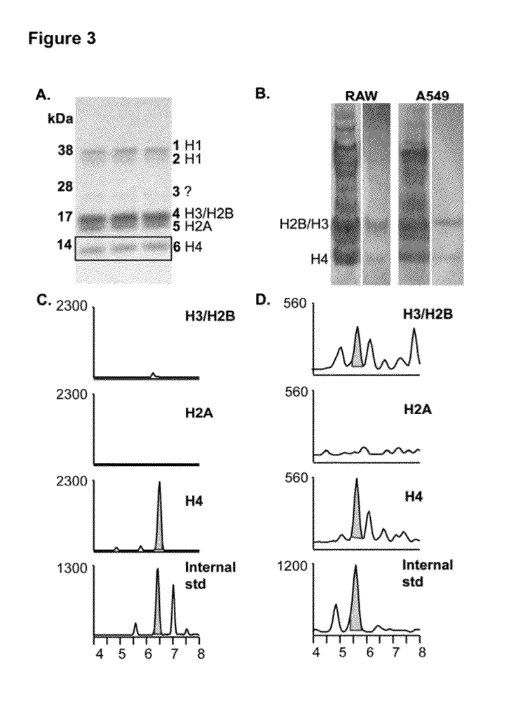

Cultured cells were lysed in hypotonic buffer (10 mM Tris/10 mM NaCl/3 mM MgCl₂) containing 1 mM pyridoxamine and 100 μM indomethacin to prevent the artifactual formation of LGE₂ during the lytic process. After letting cells swell on ice, membranes were disrupted by addition of Triton X-100 (0.5% final concentration) and vortexing. Nuclei were isolated by centrifugation for 10 min at 1000×g, and the resulting pellet washed with PBS. Histones were extracted in 0.4 N H₂SO₄, precipitated with trichloracetic acid, and washed with acetone. With this method, histones are the predominant proteins and contaminating nuclear proteins are reduced (FIG. 3A). Histones were resolubilized in dilute NaOH, and pH neutralized with HCl. Protein concentration was determined using the method of Bradford. For tissue, a portion of frozen liver was homogenized in buffer containing 40 mM sodium citrate, and 1% Triton X-100 with 3 mM Trolox and 100 μM indomethacin. The supernatant was separated from settled debris, and nuclei were pelleted at 500×g and washed. Nuclear pellets were frequently transferred to fresh tubes to avoid contamination by floating lipid debris; any remaining was removed with a cotton swab. Histones were extracted as above. All centrifugation steps were carried out at 4° C.

### Sample Preparation and Mass Spectrometry

Histone samples were prepared for mass spectrometry by addition of ammonium bicarbonate to 5 mM final concentration before digesting to single amino acids by protease step-digestion as previously described. In the case of immunoblots, proteins were digested directly off the nitrocellulose through incubation of the nitrocellulose strip in 30 μg/mL Pronase in ammonium bicarbonate buffer, and later addition of aminopeptidase. All samples were centrifuged at 2000×g for 10 min. after final digestion to remove precipitate, spiked with 0.2 ng ¹³C-lysyl-lactam internal standard, and purified on prepared tC18 cartridges (Waters Corp., Milford, Mass.). Samples on tC18 cartridges were washed with water, then 30% methanol, before being eluted in 80% methanol and concentrated by evaporation. Samples were evaluated by electrospray ionization (ESI) LC/MS/MS on a ThermoFisher TSQ Quantum triple quadrapole mass spectrometer in positive ion mode and quantitated by isotopic dilution as previously described, with the exception of a reduced flow rate of 0.1 mL/min.

### Measurement of PGE₂

A sample of cellular media was taken just prior to lysis and centrifuged to remove any cellular debris. For PGE₂ analysis, samples were spiked with 2 ng of [²H₇] PGE₂ as an internal standard. Prostaglandins were isolated and derivatized for analysis by GC/MS, operating in negative ion chemical ionization (NICI) mode and monitoring selected ions as previously described. For the [²H₄] PGE₂ internal standard, m/z=528. To account for the deuterium-protium exchange at the position C12 of [²H₇] PGE₂, the summation of the signals obtained at m/z=530, m/z=531 and m/z=532 was performed.

### Autoradiography

A549 or RAW264.7 cells were treated overnight to stimulate COX-2 expression and given 20 μM ¹⁴C-AA (116 μCi) for 1 h. Histones were isolated as described above and separated on 4-12% SDS-PAGE gels (Life Technologies), which were stained with Coomassie and exposed to film for 2 weeks.

### Salt Extraction

60-80% confluent A549 cells were stimulated and treated with AA±500 μM EtSA before scraping cells in lysis media for nuclear isolation as above. After addition of Tritox X-100 and vortexing, 1.5 mL of each sample was aliquoted into an eppendorf and centrifuged to separate nuclei. Pellets were washed with PBS and all buffer removed. Nuclear pellets were resuspended in extraction buffer containing 0.6M, 0.9M, 1.2M or 1.5M NaCl (with 10 mM Tris, pH 7.5, 3 mM MgCl₂, 0.5% NP-40, and protease inhibitor cocktail) and incubated 10 min on ice. Following this extraction period, the nuclei were centrifuged at 16,000×g to obtain the soluble fraction. This was sonicated, denatured at 95° C., and analyzed by SDS-PAGE and Western blotting, using Ponceau stain to visualize proteins and confirm equal loading, and anti-H4 antibody (Abcam, Cambridge, Mass.).

### Statistical Analyses

All data were analyzed using Prism software (GraphPad, La Jolla, Calif.). Data are expressed as means±SE, and statistical significance was determined using one-way ANOVA followed by Tukey's post-test or Dunnett's multiple comparisons post-test, when appropriate. A p value <0.05 was considered significant.

### Results

### Levuglandins Form Adducts on Histones in Cultured Cell and Whole Tissue

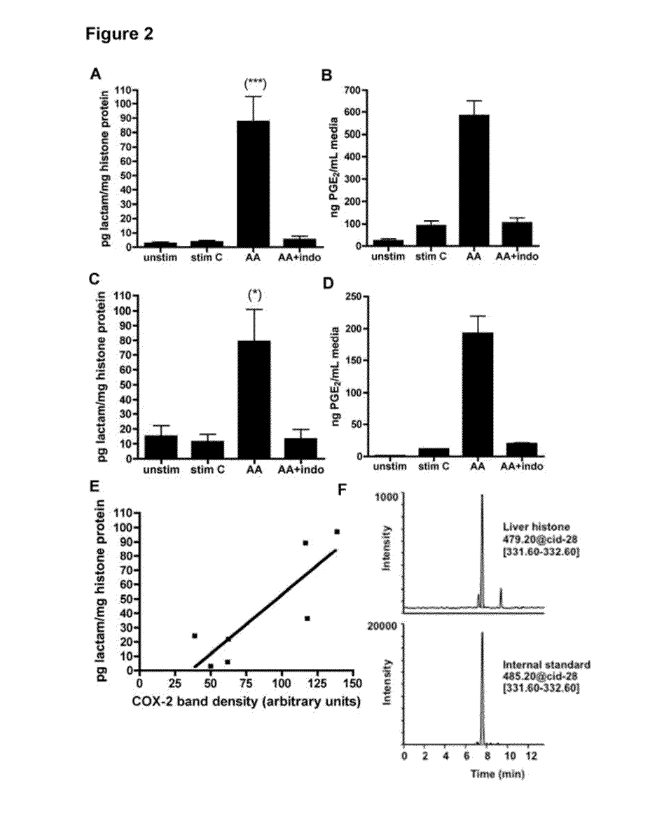

With mass spectrometry, the present inventors identified LG-lysyl adducts on histones in RAW264.7 macrophages (FIG. 2A) as well as A549 cultured lung epithelial cells (FIG. 2C). COX-2 is upregulated in these cells upon cytokine stimulation, and addition of exogenous AA leads to formation of LG-histone adducts. Formation of these adducts is blocked with indomethacin, further indicating a COX-dependent mechanism (FIGS. 2A and 2C). Very few LG-histone adducts are formed in these cell lines without addition of exogenous AA, and PGE₂ analysis of cell media from each group indicates there is comparatively little endogenous AA mobilized following induction of COX-2 (FIGS. 2B and 2D). Although few adducts are formed at basal levels in our cell lines, we find the LG-lysyl adduct in rat liver histones (FIG. 2E), where levels correlate with COX-2 expression, demonstrating COX-2-dependent adduct formation under physiological conditions.

### LG-Histone Adducts are Restricted to Specific Histone Isoforms

Using 0.4N H₂SO₄ to extract histones results in a relatively pure preparation, with histones corresponding to known molecular weights (FIG. 3A). Incubation of ¹⁴C-AA with stimulated A549 or RAW264.7 cells led to a ¹⁴C-containing band in the histone preparation that corresponded with H4 and H3/H2B, despite the fact that other histones are represented in equal or greater quantity (FIG. 3B). The present inventors treated stimulated RAW264.7 macrophages or A549 lung carcinoma cells with 20 μM AA for 1 h and directly digested and analyzed the SDS-PAGE bands as labeled in FIG. 3A. The H4 band yielded a predominant peak corresponding with the internal LG-lysyl standard, while lower or no signal was seen in the H3/H2B band and no corresponding peaks were seen in other bands (FIGS. 3C and 3D, data not shown). Thus, there is consistent evidence for formation of an LG-lysine lactam adduct on H4. The radiolabeled AA product adducted to H3/H2B probably includes structures in addition to the LG-lysine lactam. These results, from two separate cell lines, suggest that there is specificity in the reaction of LG with histones.

The Scavenger S-Ethylsalicylamine (EtSA) Reduces LG-Histone Adduct Formation without Affecting COX-2 Activity

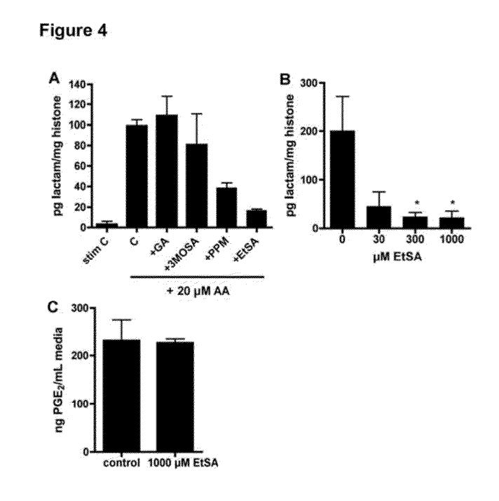

As demonstrated herein, in RAW264.7 cells, 5-ethylsalicylamine (EtSA) most effectively blocked adduct formation. Pentylpyridoxamine partially inhibited histone adduct formation, but was much less potent than 5-ethylsalicylamine (FIG. 4A). EtSA also inhibited LG-histone adduct formation in stimulated, AA-treated A549 cells, without affecting PGE₂ production at the highest concentration tested (FIGS. 4B and C).

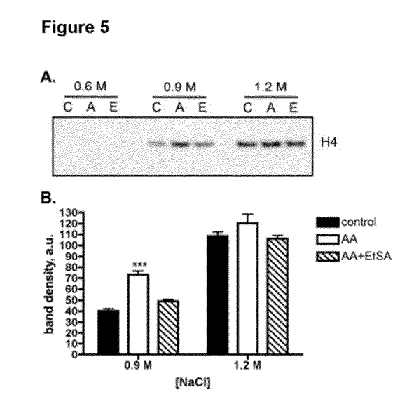

Formation of LG-lysyl adduct on histone H4 decreases DNA-histone interaction To examine the functional effect of LG-histone adduction, we performed salt fractionation of A549 nuclei to determine histone solubility. In this assay, loosely-bound histone is released at lower salt concentrations than tightly-bound proteins. The present inventors discovered that in stimulated, AA-treated A549 cells, histone H4 was eluted at lower salt concentrations than in stimulated control cells; this was reversed after treatment with the scavenger EtSA (FIG. 5).

### DISCUSSION

As indicated herein, the present inventors established that COX-2 catalysis can cause changes in DNA-histone interactions through formation of LG-histone adducts, suggests a new hypothesis for the contribution of COX-2 to the etiology of cancer. Oxidative damage is known to cause N⁶-formylation of H1 histone, and epigenetic modification affecting COX-2 transcription is well-described, but the LG-lysyl histone adduct we describe here is an entirely novel finding that links inflammation and COX-2 activation with histone modification.

The present inventors discovered COX-2 dependent formation of LG-histone adducts in cells and tissues. Whereas COX-2 blockade by treatment with indomethacin decreases LG-histone adduct formation in A549 or RAW264.7 cells, this method of antagonism cannot separate the myriad effects of other COX-2 products from the effects of the LG-histone adducts. The present inventors screened a number of small molecule levuglandin scavenger molecules for their ability to decrease LG-histone adduction. LG will react with these molecules three orders of magnitude faster than lysine, and we have previously shown that scavenger treatment decreases total cellular levels of LG-lysine adducts without affecting PGE₂ production. These scavengers are orally bioavailable, and able to decrease total LG-protein adduct levels when given to mice in drinking water. In future studies, aside from allowing investigation of LG-protein modification independent of COX activity, use of these scavengers may bypass the cardiovascular and gastric side effects seen with COX inhibitors.

Interestingly, not all histones are targeted, but cellular LG adducts seem to preferentially form on the H4 and, to a lesser extent, on H3/H2B. Whether this specificity is a reflection of histone availability in the nucleosome, accessibility of lysine residues, or a more favorable microenvironment for adduct formation remains to be shown. Incubation with [¹⁴C]-AA in cells led to a stronger autoradiographic band at H3/H2B compared to H4, while LC-MS analysis indicated that H4 was the major form adducted by LGs. This discrepancy can be explained by several mechanisms. Protein-associated radioactivity would come from any product derived from [¹⁴C] AA, including LGs but also PGJ₂/PGA₂ cyclopentenone or arachidonate ester adducts. In addition, the occurrence of LG-lysyl adducts is almost certainly underreported with our current approach. Our internal standard and mass spectroscopy method are specific for detection of a single LG-lysyl adduct with an m/z equivalent to the lactam structure, but the initial Schiff base intermediate of LG-lysyl adducts can oxidize to form other structure such as hydroxylactam or intraprotein or protein-DNA crosslinks, which would go undetected in our method. For these reasons, the autoradiograms can not be quantitatively compared with the LC/ESI/MS/MS results as they do not measure the same molecular structures.

H4, along with H2A, H2B, and H3 histones, comprise the histone octamer around which DNA is “packaged” into nucleosomes. The interaction of histone N-terminal tails with DNA is critical to DNA compaction and organization, and is dependent on the numerous positively charged lysine and arginine residues present; a mesoscopic model demonstrates that H4 tails are the most important in mediating internucleosomal interactions. A single lysyl acetylation on K16 of H4 modulates chromatin compaction and interaction of numerous chromatin-associated proteins; constitutive acetylation of this residue confers a folding defect comparable to deletion of the entire H4 tail. After LG-histone adduct formation we do find disruption of histone-DNA binding, resulting in increased DNA extraction in a salt solution. This decreased histone-DNA interaction may increase DNA transcriptional access to previously silent oncogenes, and contribute to the development of cancer.

The complex patterns of lysyl acetylation and methylation comprise a “histone code” that regulates chromatin access and transcription; it is plausible that irreversible adduction of lysyl residues could disrupt this code, or directly alter the access of DNA-interacting proteins. Changes in histone modifications are known to result in altered DNA methylation, deregulation of oncogenes, genomic instability, impaired DNA repair, and defects in cell cycle checkpoints. Changes in lysyl modifications of H4 in particular are a common hallmark of human cancers, and are associated with a global loss of DNA methylation. Further elucidation of the effects of LG-histone adduction on histone modification, DNA-histone interactions, and transcription should increase our understanding of the molecular mechanisms whereby COX-2 contributes to cancer development and progression.

### REFERENCES

The following references are incorporated herein by reference in their entirety:
1. Agrawal, A., and Fentiman, I. S. (2008) NSAIDs and breast cancer: a possible prevention and treatment strategy, Int J Clin Pract 62, 444-449.
2. Harris, R. E., Beebe-Donk, J., and Alshafie, G. A. (2007) Reduced risk of human lung cancer by selective cyclooxygenase 2 blockade: results of a case control study, Int J Biol Sci 3, 328-334.
3. Konturek, P. C., Kania, J., Burnat, G., Hahn, E. G., and Konturek, S. J. (2005) Prostaglandins as mediators of COX-2 derived carcinogenesis in gastrointestinal tract, J Physiol Pharmacol 56 Suppl 5, 57-73.
4. Harris, R. E. (2009) Cyclooxygenase-2 (cox-2) blockade in the chemoprevention of cancers of the colon, breast, prostate, and lung, Inflammopharmacology 17, 55-67.
5. Lim, B. J., Jung, S. S., Choi, S. Y., and Lee, C. S. (2010) Expression of metastasis-associated molecules in non-small cell lung cancer and their prognostic significance, Mol Med Report 3.43-49.
6. Sheehan, K. M., Sheahan, K., O'Donoghue, D. P., MacSweeney, F., Conroy, R. M., Fitzgerald, D. J., and Murray, F. E. (1999) The relationship between cyclooxygenase-2 expression and colorectal cancer, JAMA 282, 1254-1257.
7. Bertagnolli, M. M. (2007) Chemoprevention of colorectal cancer with cyclooxygenase-2 inhibitors: two steps forward, one step back, Lancet Oncol 8, 439-443.
8. Castellone, M. D., Teramoto, H., Williams, B. O., Druey, K. M., and Gutkind, J. S. (2005) Prostaglandin E2 promotes colon cancer cell growth through a Gs-axin-beta-catenin signaling axis, Science 310, 1504-1510.
9. Nakanishi, M., Montrose, D. C., Clark, P., Nambiar, P. R., Belinsky, G. S., Claffey, K. P., Xu, D., and Rosenberg, D. W. (2008) Genetic deletion of mPGES-1 suppresses intestinal tumorigenesis, Cancer research 68, 3251-3259.
10. Elander, N., Ungerbäck, J., Olsson, H., Uematsu, S., Akira, S., and Söderkvist, P. (2008) Genetic deletion of mPGES-1 accelerates intestinal tumorigenesis in APC Min/+ mice, Biochemical and biophysical research communications 372, 249-253.
11. Boutaud, O., Brame, C. J., Salomon, R. G., Roberts, L. J., 2nd, and Oates, J. A. (1999) Characterization of the lysyl adducts formed from prostaglandin H12 via the levuglandin pathway, Biochemistry 38, 9389-9396.
12. Salomon, R. G., Miller, D. B., Zagorski, M. G., and Coughlin, D. J. (1984) Solvent-induced fragmentation of prostaglandin endoperoxides. New aldehyde products from PGH2 and a novel intramolecular 1,2-hydride shift during endoperoxide fragmentation in aqueous solution, J Am Chem Soc 106, 6049-6060.
13. Zagol-Ikapitte, I., Bernoud-Hubac, N., Amamath, V., Roberts, L. J., 2nd, Boutaud, O., and Oates, J. A. (2004) Characterization of bis(levuglandinyl) urea derivatives as products of the reaction between prostaglandin H2 and arginine, Biochemistry 43, 5503-5510.
14. Boutaud, O., Brame, C. J., Chaurand, P., Li, J., Rowlinson, S. W., Crews, B. C., Ji, C., Marnett, L. J., Caprioli, R. M., Roberts, L. J., 2nd, and Oates, J. A. (2001) Characterization of the lysyl adducts of prostaglandin H-synthases that are derived from oxygenation of arachidonic acid, Biochemistry 40, 6948-6955.
15. Iyer, R. S., Ghosh, S., and Salomon, R. G. (1989) Levuglandin E2 crosslinks proteins, Prostaglandins 37, 471-480.
16. Boutaud, O., Li, J., Zagol, I., Shipp, E. A., Davies, S. S., Roberts, L. J., 2nd, and Oates, J. A. (2003) Levuglandinyl adducts of proteins are formed via a prostaglandin H2 synthase-dependent pathway after platelet activation, The Journal of biological chemistry 278, 16926-16928.
17. Boutaud, O., Andreasson, K. I., Zagol-Ikapitte, I., and Oates, J. A. (2005) Cyclooxygenase-dependent lipid-modification of brain proteins, Brain Pathol 15, 139-142.
18. Jenuwein, T., and Allis, C. D. (2001) Translating the histone code, Science 293, 1074-1080.
19. Esteller, M. (2007) Cancer epigenomics: DNA methylomes and histone-modification maps, Nat Rev Genet 8, 286-298.
20. Fraga, M. F., Ballestar, E., Villar-Garea, A., Boix-Chornet, M., Espada, J., Schotta, G., Bonaldi. T., Haydon, C., Ropero, S., Petrie, K., Iyer, N. G., Perez-Rosado, A., Calvo, E., Lopez, J. A., Cano, A., Calasanz, M. J., Colomer, D., Piris, M. A., Ahnim, N., Imhof, A., Caldas, C., Jenuwein, T., and Esteller, M. (2005) Loss of acetylation at Lys16 and trimethylation at Lys20 of histone H4 is a common hallmark of human cancer, Nat Genet 37, 391-400.
21. Zagol-Ikapitte, I., Amarnath, V., Bala, M., Roberts, L. J., 2nd, Oates, J. A., and Boutaud, O. (2010) Characterization of scavengers of gamma-ketoaldehydes that do not inhibit prostaglandin biosynthesis, Chemical research in toxicology 23, 240-250.
22. Zagol-Ikapitte, I., Masterson, T. S., Amarnath, V., Montine, T. J., Andreasson, K. I., Boutaud, O., and Oates, J. A. (2005) Prostaglandin H(2)-derived adducts of proteins correlate with Alzheimer's disease severity, Journal of neurochemistry 94, 1140-1145.
23. Shechter, D., Dormann, H. L., Allis, C. D., and Hake, S. B. (2007) Extraction, purification and analysis of histones, Nat Prot 2, 1445-1457.
24. Bhaskara, S., Knutson, S. K., Jiang, G., Chandrasekharan, M. B., Wilson, A. J., Zheng, S., Yenamandra, A., Locke, K., Yuan, J-L., Bonine-Summers, A. R., Wells, C. E., Kaiser, J. F., Washington, M. K., Zhao, Z., Wagner, F. F., Sun, Z.-W., Xia, F., Holson, E. B., Khabele, D., and Hiebert, S. W. (2010) Hdac3 is essential for the maintenance of chromatin structure and genome stability, Cancer Cell 18, 436-447.
25. Sanders, M. M. (1978) Fractionation of nucleosomes by salt elation from micrococcal nuclease-digested nuclei, J Cell Biol 79, 97-109.
26. Jiang, T., Zhou, X., Taghizadeh, K., Dong, M., and Dedon, P. C. (2007) N-formylation of lysine in histone proteins as a secondary modification arising from oxidative DNA damage, Proceedings of the National Academy of Sciences of the United States of America 104, 60-65.
27. Fernandez-Alvarez, A., Llorente-Izquierdo, C., Mayoral, R., Agra, N., Bosca, L., Casado, M., and Martin-Sanz, P. (2012) Evaluation of epigenetic modulation of cyclooxygenase-2 as a prognostic marker for hepatocellular carcinoma, Oncogenesis 1, e23.
28. Amarnath, V., Amarnath, K., Davies, S., and Roberts, L. J. n. (2004) Pyridoxamine: an extremely potent scavenger of 1,4-dicarbonyls, Chemical research in toxicology 17, 410-415.
29. Zagol-Ikapitte, I., Amarnath, V., Jadhav, S., Oates, J. A., and Boutaud, O. (2011) Determination of 3-methoxysalicylamine levels in mouse plasma and tissue by liquid chromatography-tandem mass spectrometry: application to in vivo pharmacokinetics studies, Journal of chromatography 879, 1098-1104.
30. Zagol-Ikapitte, I. A., Matafonova, E., Amarnath, V., Bodine, C. L., Boutaud, O., Tirona, R. G., Oates, J. A., Roberts, L. J., 2nd, and Davies, S. S. (2010) Determination of the pharmacokinetics and oral bioavailability of salicylamine, a potent gamma-ketoaldehyde scavenger, by LC/MS/MS, Pharmaceutics 2, 18-29.
31. Murthi, K. K., Friedman, L. R., Oleinick, N. L., and Salomon, R. G. (1993) Formation of DNA-protein cross-links in mammalian cells by levuglandin E2, Biochemistry 32, 4090-4097.
32. Arya, G., and Schlick, T. (2006) Role of histone tails in chromatin folding revealed by a mesoscopic oligonucleosome model, Proceedings of the National Academy of Sciences of the United States of America 103, 16236-16241.
33. Shogren-Knaak, M., Ishii, H., Sun, J.-M., Pazin, M. J., Davie, J. R., and Peterson, C. L. (2006) Histone H4-K16 acetylation controls chromatin structure and protein interactions, Science 311, 844-847.
34. Fuks, F. (2005) DNA methylation and histone modifications: teaming up to silence genes, Curr Opin Genet Dev 15, 490-495.
35. Fiillgrabe, J., Kavanagh, E., and Joseph, B. (2011) Histone onco-modifications, Oncogene 30, 3391-3403.

We claim:

1. A method of inhibiting formation of levuglandin adducts of histone and DNA in a subject in need thereof by administering a levuglandin adduct formation inhibiting amount of a compound of the following formula: 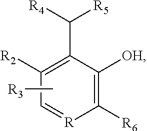
wherein:
R is N or CR₄;
R₂ is H, substituted or unsubstituted alkyl, substituted or unsubstituted alkoxy, alkyl-alkoxy-R₄;
R₃ is H, halogen, substituted or unsubstituted alkyl, substituted or unsubstituted alkoxy, hydroxyl, nitro;
R₄ is a bond, H, substituted or unsubstituted alkyl, substituted or unsubstituted alkoxy, carboxyl, substituted or unsubstituted aryl, or substituted or unsubstituted cycloalkyl;
R₅ is amine;
R₆ is H, substituted or unsubstituted alkyl, substituted or unsubstituted alkoxy;
or analogs thereof, and pharmaceutical salts thereof.

2. The method of claim 1, wherein the compound is of the following formula: 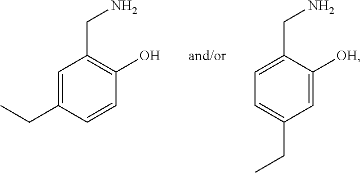
or a pharmaceutical salt thereof.

3. A method of treating pre-malignant lesions by administering an effective amount of a compound of the following formula: 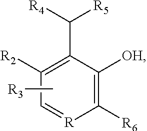
wherein:
R is N or CR₄;
R₂ is H, substituted or unsubstituted alkyl, substituted or unsubstituted alkoxy, alkyl-alkoxy-R₄;
R₃ is H, halogen, substituted or unsubstituted alkyl, substituted or unsubstituted alkoxy, hydroxyl, nitro;
R₄ is a bond, H, substituted or unsubstituted alkyl, substituted or unsubstituted alkoxy, carboxyl, substituted or unsubstituted aryl, or substituted or unsubstituted cycloalkyl;
R₅ is amine;
R₆ is H, substituted or unsubstituted alkyl, substituted or unsubstituted alkoxy;
or analogs thereof; and pharmaceutical salts thereof.

4. The method of claim 3, wherein the compound is of the following formula: 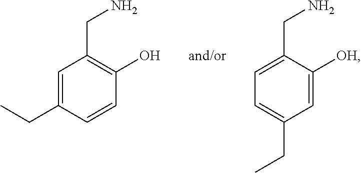
or a pharmaceutical salt thereof.

5. The method of claim 3, wherein the lesion exists in the colon, esophagus, breast, lung, pancreas and/or prostate.

6. The method of claim 1, wherein said inhibition lowers levels of LC-histone adduct formation in said subject.

7. The method of claim 6, wherein said lowered level of LG-histone adduct formation treats or prevents a disorder resulting from elevated levels of LG-histone adduct formation.

8. The method of claim 7, wherein the disorder resulting from elevated levels of LG-histone adduct formation is neoplasia.

9. The method of claim 8, wherein the neoplasia is a brain cancer, a bone cancer, an epithelial cell-derived neoplasia (epithelial carcinoma), a basal cell carcinoma, an adenocarcinoma, a gastrointestinal cancer, a lip cancer, a mouth cancer, an esophageal cancer, a small bowel cancer, a stomach cancer, a colon cancer, a liver cancer, a bladder cancer, a pancreas cancer, an ovary cancer, a cervical cancer, a lung cancer, a breast cancer, a skin cancer, a squamus cell cancer, a basal cell cancer, a prostate cancer, a renal cell carcinoma, a cancerous tumor, a growth, a polyp, an adenomatous polyp, a familial adenomatous polyposis or a fibrosis resulting from radiation therapy.

10. The method of claim 1, wherein the compound is of the following formula: 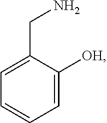
or an analog thereof, and pharmaceutical salts thereof.

11. The method of claim 1, wherein the compound is of the following formula: 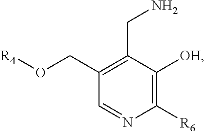
or an analog thereof, and pharmaceutical salts thereof.

12. The method of claim 1, wherein the compound is of the following formula: 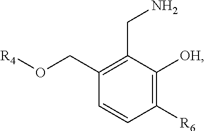
or an analog thereof, and pharmaceutical salts thereof.

13. The method of claim 1, wherein the compound is of the following formula: 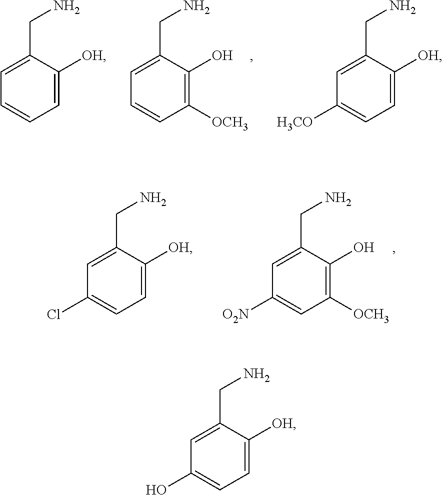
or an analog thereof.

14. The method of claim 1, wherein the compound is of the following formula: 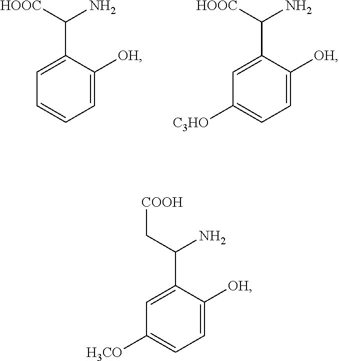
or an analog thereof.

15. The method of claim 1, wherein the compound is of the following formula:

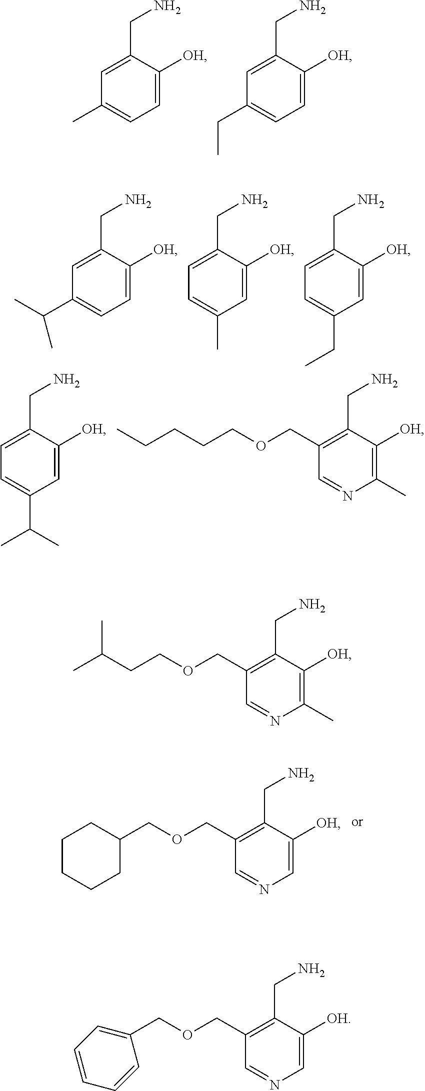

16. The method of claim 3, wherein the compound is of the following formula: 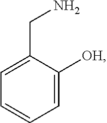
or an analog thereof, and pharmaceutical salts thereof.

17. The method of claim 3, wherein the compound is of the following formula: 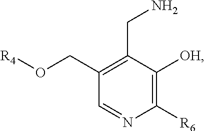
or an analog thereof, and pharmaceutical salts thereof.

18. The method of claim 3, wherein the compound is of the following formula: 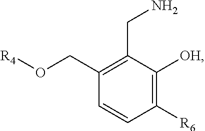
or an analog thereof, and pharmaceutical salts thereof.

19. The method of claim 3, wherein the compound is of the following formula: 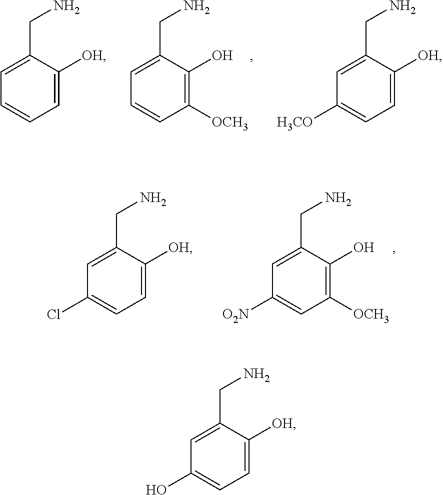
or an analog thereof.

20. The method of claim 3, wherein the compound is of the following formula: 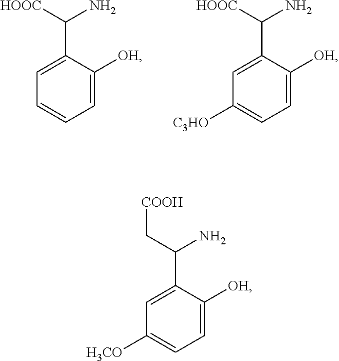
or an analog thereof.

21. The method of claim 3, wherein the compound is of the following formula: [chemical structure]
